(19) United States
(12) Patent Application Publication
(10) Pub. No.: US 2009/0098587 A1
(43) Pub. Date: Apr. 16, 2009

(54) METHOD OF SCREENING FOR DISORDERS OF GLUCOSE METABOLISM

(75) Inventors: Donald Hetzel, Phoenix, AZ (US); Stephen L. Monfre, Gilbert, AZ (US); Kevin H. Hazen, Gilbert, AZ (US); Timothy L. Ruchti, Gilbert, AZ (US); Thomas B. Blank, Chandler, AZ (US); Linda Hockersmith, Scottsdale, AZ (US); Andrew Cone, Phoenix, AZ (US)

Correspondence Address: GLENN PATENT GROUP, 3475 EDISON WAY, SUITE L, MENLO PARK, CA, 94025, US

(73) Assignee: SENSYS MEDICAL, INC., Arizona, AZ (US)

(21) Appl. No.: 12/099,740

(22) Filed: Apr. 8, 2008

Related U.S. Application Data
(63) Continuation of application No. 10/702,236, filed on Nov. 5, 2003, now abandoned.
Application No. 10/702,236 is a continuation-in-part of application No. 10/219,200, filed on Aug. 13, 2002, now Pat. No. 7,395,158.
(60) Provisional application No. 60/312,155, filed on Aug. 13, 2001.

(51) Int. Cl. C12Q 1/54

(52) U.S. Cl. 435/14

(57) ABSTRACT

A method of screening for disorders of glucose metabolism such as impaired glucose tolerance and diabetes allows prevention, or early detection and treatment of diabetic complications such as cardiovascular disease, retinopathy, and other disorders of the major organs and systems. A mathematical algorithm evaluates the shape of a subject's glucose profile and classifies the profile into one of several predefined clusters, each cluster corresponding either to a normal condition or one of several abnormal conditions. The series of blood glucose values making up the glucose tolerance curve may be measured using any glucose analyzer including: invasive, minimally invasive and noninvasive types. The method is executed on a processing device programmed to perform the steps of the method. Depending on the outcome of the screening, a subject may be provided with additional information concerning their condition and/or counseled to consult further with their health care provider.

### CROSS-REFERENCES TO RELATED APPLICATIONS

This application:
is a continuation of U.S. patent application Ser. No. 10/702,236 filed Nov. 5, 2003, which
   is a continuation-in-part of U.S. patent application Ser. No. 10/219,200, filed on Aug. 13, 2002, which claims benefit of U.S. provisional patent application Ser. No. 60/312,155, filed on Aug. 13, 2001; and
   claims benefit of U.S. provisional patent application Ser. No. 60/424,481, filed on Nov. 6, 2002; and
   claims benefit of U.S. provisional patent application Ser. No. 60/425,780, filed on Nov. 12, 2002, all of which are incorporated herein in their entirety by this reference thereto.

### BACKGROUND OF THE INVENTION

1. Field of the Invention

The invention relates generally to measurement of blood and tissue analytes. More particularly the invention relates to a method of screening for disorders of glucose metabolism.

2. Background Information

Diabetes is a chronic and incurable disease in which the body does not produce or properly use insulin, a hormone that allows glucose to enter the cells of the body and be utilized for energy. The cause of diabetes is not yet known, although both genetic and environmental factors such as obesity and lack of exercise appear to play roles. People with diabetes have increased risk of cardiovascular disease as well as retinopathy and neuropathy. It has been shown that tight control of glucose levels in the diabetic population to normoglycemic or slightly hyperglycemic levels results in delayed onset and slowed progression of retinopathy, nephropathy, and neuropathy [See DCCT study group, The New England Journal of Medicine, 341:1306:1309 (1993)].

With inadequate insulin utilization, glucose builds in the bloodstream instead of transporting into cells. The body is unable to use glucose for energy despite the increasing levels of glucose circulating in the blood. Initial glucose elevations may cause no symptoms. Later, the elevations may cause symptoms of fatigue, excessive thirst, urination, and hunger. These symptoms are nondescript and are often not reported to health care providers. Many people have unknown elevations for years without proper management of the disease because current diagnostic test procedures were either not ordered or not opportune during the health care visit.

There are three major types of diabetes: (Type I, Type II, and Gestational)

### Type I—Insulin Dependent Diabetes Mellitus (IDDM)—Also Known as Juvenile-Onset Diabetes

Type I diabetes is an autoimmune disease in which the body's own immune system destroys the pancreatic cells which produce insulin. This disease can occur at any age, but most often occurs in people under thirty years of age. Type I diabetes accounts for approximately ten percent of all diabetics. Presentation of symptoms is usually severe and develops rapidly. People with this condition require daily doses of insulin to stay alive. Although the exact cause of Type I diabetes is unknown, genetics, viruses that injure the pancreas, and destruction of insulin-making cells by the body's immune system may play causative roles.

### Type II—Non-Insulin Dependent Diabetes Mellitus (NIDDM)—Also Known as Adult-Onset Diabetes

Type II diabetes usually occurs due to a metabolic disorder known as insulin resistance, an inability to properly use insulin combined with relative insulin deficiency. This form of diabetes is the most common form of diabetes, accounting for approximately ninety percent of cases. People in the following categories are at a higher risk of developing Type II diabetes:
Over age forty-five;
Family history of diabetes;
Overweight;
Lack of regular exercise;
Low HDL cholesterol
High triglycerides;
Certain racial and ethnic groups; and
Women who have had gestational diabetes.

### Gestational Diabetes

According to the American Diabetes Association, Gestational diabetes mellitus (GDM) is defined as glucose intolerance with onset or first recognition during pregnancy, whether or not the condition persists after pregnancy. It does not exclude the possibility that unrecognized glucose intolerance may have antedated or begun concomitantly with the pregnancy [See http://care.diabetesjournals.org/cgi/content/full/25/suppl_{—}1/s94].

Risk assessment for GDM should be undertaken at the first prenatal visit with testing undertaken at 24-28 weeks of gestation for those at high risk:
Age >25 years;
Overweight or obese;
Member of an ethnic group with a high prevalence of GDM;
Family history of diabetes;
History of stillbirth or high birth weight infants; or
Previous gestational diabetes.

### Diabetes Prevalence and Trends

Approximately seven percent of all pregnancies are complicated by GDM, resulting in more than two hundred thousand cases annually. The prevalence may range from one to fourteen percent of all pregnancies, depending on the population studied and the diagnostic tests employed.

The World Health Organization estimates that diabetes currently afflicts one hundred fifty-four million people worldwide, fifty-four million of who live in developed countries. They also predict that the number of people with diabetes worldwide will grow to three hundred million by 2025.

As many as 15.7 million Americans, or 5.9% of the population, have diabetes with approximately 5.4 million of these people being undiagnosed. The number of Americans with diabetes has recently been estimated to be growing at a rate of nine percent per year.

In the United States, the prevalence of adults with diagnosed diabetes increased by six percent in 1999 and rose thirty-three percent nationally between 1990 and 1998. There are approximately eight hundred thousand new cases every year in America.

The risk for Type II diabetes increases with age. An estimated eighteen percent of the American population aged sixty-five and older has diabetes.

In addition to millions of Americans who suffer from diabetes, it is estimated that an additional twenty to thirty million Americans suffer from Impaired Glucose Tolerance (IGT). Approximately twenty-five percent of the American population aged sixty-five and older suffers from IGT.

### Impaired Glucose Tolerance

It is estimated that eleven percent of the American public has this condition. Impaired glucose tolerance may be viewed as an intermediate condition between normal glucose metabolism and type II diabetes. Impaired glucose tolerance, also known as pre-diabetes, is a condition in which blood sugar levels are higher than normal, but do not meet the diagnostic criteria for diabetes. Persons with IGT have a five-fold risk of developing diabetes within five years. However, the Diabetes Prevention Study has shown that early detection and intervention may delay or prevent the onset of diabetes. It also has recently been discovered that IGT individuals are at higher risk for cardiovascular disease and death, a risk evaluated in the Whitehall Study, the Paris Prospective Study, and the Helsinki Policeman Study [See Diabetes Care, 21:360-367 (1998)] and discovered to be greater than in people with diabetes. It is reasonable to suppose that with the early detection and treatment of IGT, strategies to mitigate cardiovascular risk as well as diabetes prevention may be pursued. Prevention or early treatment of diabetes would have the added benefit of reducing diabetic complications such as kidney disease, nerve disease, blindness, diabetic ketoacidosis, and a shorter lifespan. For these reasons, early detection of IGT is critical to the general health of our population.

### Hyperinsulinemia (Postprandial Reactive Hypoglycemia)

Postprandial reactive hypoglycemia is a medical condition in which symptoms occur after a meal as a response to food stimulation as opposed to a fasting state. Blood sugar levels are normally around 90 to 110 mg/dL, but with hypoglycemia they are usually below 50 mg/dL and may get as low as 35 mg/dL.

There are two reasons for the symptoms: (1) adrenaline release and (2) glucose deprivation of the nervous system. Low blood sugar stimulates the release of adrenaline, which causes shakiness, sweating, hunger pangs, nervousness, and irritability. The brain doesn't get enough sugar, and commonly reported symptoms are headache, mental dullness, and fatigue. If the blood sugar drops too low, a person can get confused, have visual problems, develop a seizure, or even become unconscious.

It is theorized that the cause of the abnormal response stems from first phase vs. second phase insulin release mechanisms in the pancreas. First phase release is diminished allowing a rapid increase in blood glucose levels. It is followed by an over-responsive second phase release causing a dramatic drop in glucose to hypoglycemic levels. Some people with reactive hypoglycemia go on to develop diabetes.

### Adverse Clinical Effects of Diabetes and Impaired Glucose Tolerance

Diabetes and impaired glucose tolerance have been called “silent killers” because many people are unaware that they have the disease until they develop one of its life-threatening complications. Complications of diabetes include retinopathy, neuropathy, and cardiovascular problems [http://www.diabetes.org:80/main/application/commercewf?origin=*.jsp&event=link(B1)].

Heart Disease and Stroke: People with diabetes are two to four times more likely to have heart disease or suffer a stroke. Additionally, heart disease is present in seventy-five percent of diabetes-related deaths.

Kidney Disease: Long-term hyperglycemia results in the kidneys filtering excess blood. This extra work results in small leaks. Protein is lost into the urine. A small amount of protein in the urine is microalbuminuria while a larger concentration is proteinuria or macroalbuminuria. The overwork also diminishes the filtering capacity of the kidneys, ultimately leading to end-stage renal disease. While not everyone who has diabetes develops kidney disease, diabetes is the leading cause of end-stage renal disease, accounting for about forty percent of new cases each year. Between ten and twenty percent of all diabetics develop kidney disease due to diabetic nephropathy and require dialysis or a kidney transplant in order to stay alive.

Neuropathy (Nerve Disease and Amputations): A common complication of diabetes is diabetic neuropathy, which is a group of nerve diseases affecting peripheral nerves especially those of the fingertips and toes. Roughly two-thirds of diabetics have some form of neuropathy with symptoms ranging from loss of sensation in the feet to lower limb amputation due to unnoticed infections. Each year, fifty-six thousand Americans lose a lower limb to diabetes.

Retinopathy: Retinopathy includes all abnormalities of the small blood vessels of the retina caused by diabetes. Most diabetics have nothing more than minor eye disorders related to their diabetes. However, diabetes is the leading cause of new cases of blindness among those aged twenty to seventy-four years with twelve thousand to twenty-four thousand new blindness cases due to diabetic retinopathy occurring each year. Overall, people with diabetes have a higher risk of blindness. Early detection and treatment of diabetes can reduce the risk of blindness in many patients.

Diabetic Ketoacidosis (DKA): One of the most serious outcomes of poorly controlled diabetes, DKA is marked by high blood glucose levels along with ketones in the urine and occurs primarily in Type I individuals. DKA is responsible for about ten percent of diabetes-related deaths in individuals under age forty-five.

Skin Conditions: Diabetes may also affect the skin. Up to one third of diabetics may have a skin disorder during some part of their life. Skin problems that occur primarily with diabetics are dermopathy, necrobiosis lipoidica diabeticorum, diabetic blisters, and eruptive xanthomatosis.

Gum Disease: There is an increased risk in diabetics of developing periodontal disease. Excess circulatory glucose contributes to bacterial plaque formation.

Shorter Lifespan: Life expectancy of people with diabetes averages fifteen years less than people without the disease. Diabetes is the seventh leading cause of death in the United States, contributing to approximately two hundred thousand deaths per year.

Impotence: Males are more likely to experience impotence due to changes or disturbances in the peripheral nervous system (neuropathy) or blood vessel blockage. Impotence affects approximately thirteen percent of men with Type I diabetes and eight percent of men with Type II diabetes.

Fetal Complications: Infants of gestationally diabetic mothers are at higher risk of fetal anomalies, e.g. birth defects, macrosomia, higher birth weights, postpartum hypoglycemia, and respiratory distress syndrome [http://www.diabetes.org:80/main/application/commercewf?origin=*.jsp&event=link(B1)].

In view of the above, there exists a great need in the art for a rapid, convenient, and economical method for routine and early detection of disorders of glucose metabolism.

### DESCRIPTION OF RELATED TECHNOLOGY

Current screening tests for disorders of glucose metabolism are sub-optimal.

Screening tests often utilize glucose determinations at a few select time periods such as during fasting or two hours postprandial. These discrete tests often fail to diagnose diabetes, IGT, or even insulin resistance syndrome. People with insulin resistance syndrome are able to produce enough insulin to maintain non-diabetic glucose levels, but are still at significant risk for heart attack or stroke. Two glucose tolerance test profiles are presented in FIG. 1. The first subject glucose profile 101 has a 2-hour glucose concentration of 134 mg/dL, respectively. Under the current American Association of Clinical Endocrinologists (AACE) guideline for the 120-minute post-glucose challenge this subject is not classified as being diabetic, having IGT, or having insulin resistance syndrome despite having a peak glucose concentration of 210 mg/dL [http://www.aace.com/pub/BMI/findings.php]. Similarly, the second subject glucose profile 102 has a 2-hour concentration of 127 mg/dL. Again this subject fails the AACE guideline for even insulin resistance syndrome despite having apparent IGT based upon the peak glucose concentration of 178 mg/dL. Fasting plasma glucose levels have also been reported to fail to identify 90% of IGT and 62% of diabetes cases [Constantine Tsigo et. al. Poster 880-P, ADA 61^{st} Scientific Sessions, PA, Jun. 22-26, 2001].

### SUMMARY OF THE INVENTION

The invention provides a method of screening for disorders of glucose metabolism such as impaired glucose tolerance and diabetes, thereby allowing early treatment of the condition and possibly enabling prevention, or early detection and treatment of common complications such as cardiovascular disease, retinopathy, and other disorders of the major organs and systems.

A mathematical algorithm evaluates the shape of a subject's blood glucose profile before and after a glucose challenge and classifies the profile into one of several predefined classes, each class corresponding either to a normal condition or one of several abnormal conditions. Evaluation of the shape of the profile is accomplished through examination of one or more parameters of the profile. One embodiment of the invention provides a simple algorithm that directly compares parameters to established thresholds and ranges for the various conditions. A further embodiment of the invention provides an algorithm that characterizes a continuum of glucose concentrations or values. For example, the continuum algorithm computes a screening factor. The screening factor is then compared with thresholds determined from common diagnostic criteria. Preferably, the time series of blood glucose concentrations making up the glucose tolerance curve is measured using a noninvasive glucose analyzer, however any type of glucose analyzer, including minimally invasive and invasive devices, is suitable for practice of the invention. The values need not be actual values, relative values are also suitable, because the invention evaluates the shape of the profile, which can be discerned based on relative values. Additionally, the continuum algorithm can evaluate the profile even if parameters are missing. In addition, missing data can be supplied from historical data.

In an alternate embodiment a pattern recognition system is employed for the analysis of a glucose profile associated with a particular patient's OGTT (oral glucose tolerance test) to screen for disorders of glucose metabolism.

A processing device specifically programmed to perform the method's steps accomplishes the evaluation and classification. Depending on the outcome of the screening, a subject may be provided with additional information concerning their condition and/or counseled to consult further with their health care provider.

### BRIEF DESCRIPTION OF THE DRAWINGS

FIG. 1 indicates how current diagnostic criteria for diabetes may be misleading;

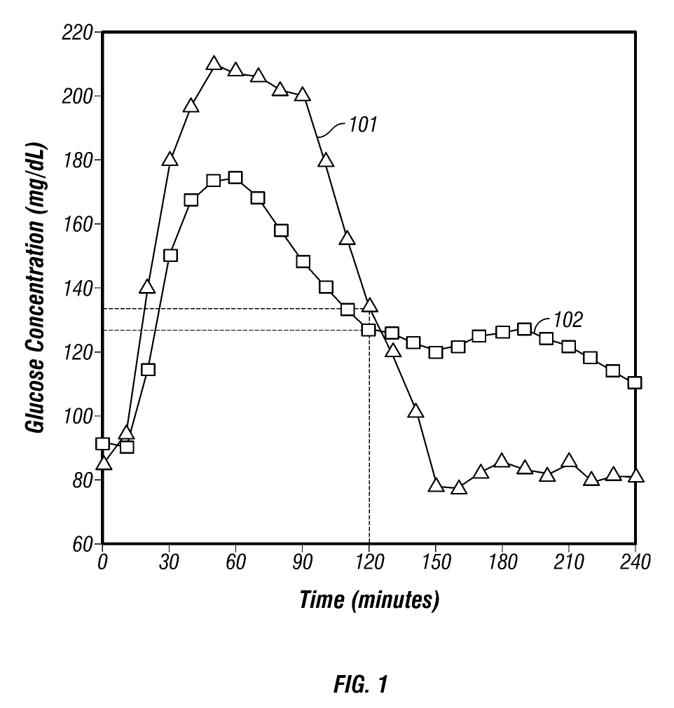

FIG. 2 shows blood glucose concentration curves for normal glucose tolerance, impaired glucose tolerance, diabetes, and hyperinsulinemia;

FIG. 3 indicates a variety of parameters on a blood glucose profile that are used to evaluate the profile according to the invention; and

FIG. 4 indicates an area under the curve for a subject having a normal blood glucose concentration curve.

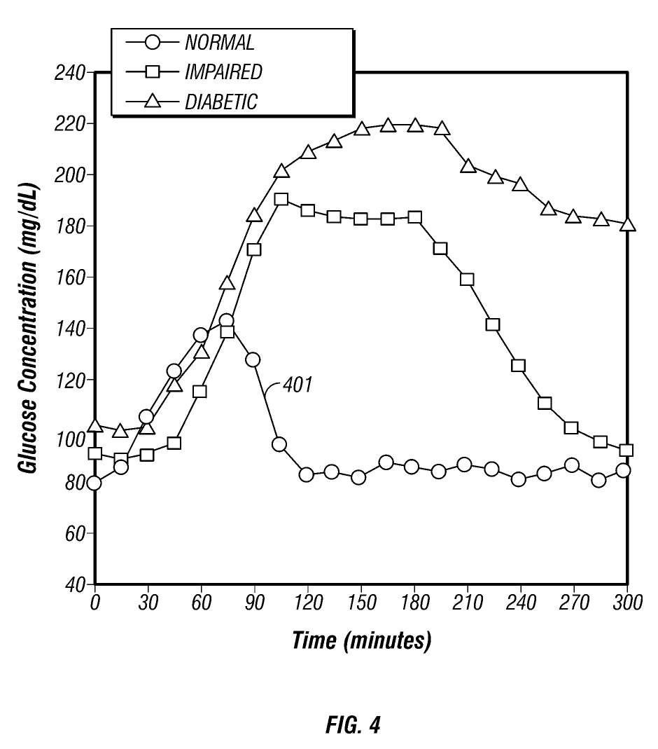

### DETAILED DESCRIPTION

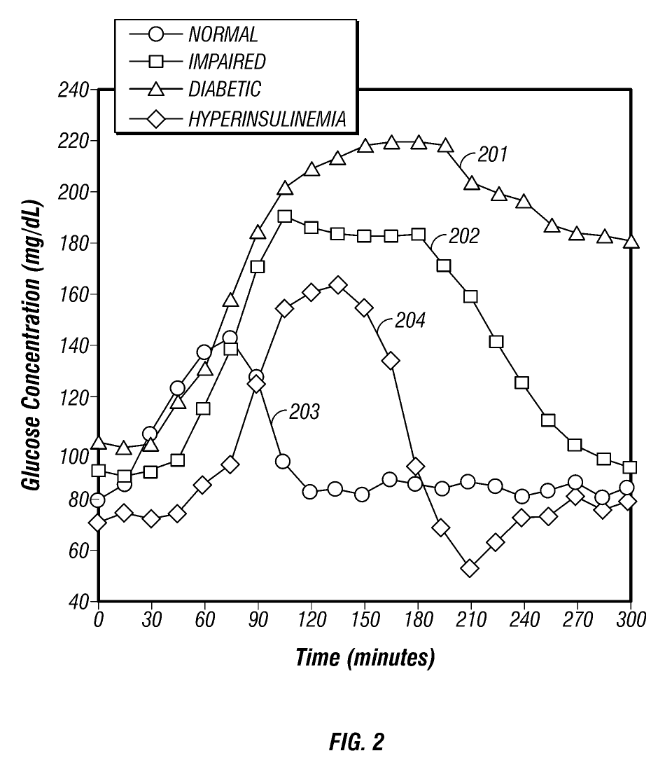

Glucose tolerance tests are well known and may be used to test a variety of disorders of glucose metabolism and hormone secretory disorders. Basically, glucose is ingested in the form of a high glucose concentration beverage or as a carbohydrate rich food. Glucose concentrations are then monitored periodically (often every hour) for a period of three to five hours, depending upon the suspected diagnostic endpoint. The shape of the glucose profile of the resulting data set may then be utilized to further identify the medical condition. For example, diabetes is diagnosed based upon the overall increase in glucose concentration from the initial fasting condition and the amount of time required for the glucose concentration to drop to a normal physiological glucose concentration of 80-120 mg/dL. According to the invention, the glucose response profile shape as a function of time relative to a glucose challenge is utilized as input data to an algorithm that first evaluates the profile and classifies it; and then outputs a screening response indicating that the subject being tested either has diabetes, IGT (impaired glucose tolerance), a normal physiological response, or abnormally low glucose tolerance (LGT). The input concentrations may be those of blood glucose determinations collected once every ten to sixty minutes. In keeping with the object of providing a convenient, inexpensive screening method, it is preferable that the glucose measurements be made with a non-invasive analyzer, however minimally invasive and invasive devices are entirely suitable for practice of the invention. FIG. 2 shows representative glucose concentration profiles for a diabetic 201, a subject with IGT 202, a subject with a normal physiological glucose response 203, and a low glucose response 204 as a function of time. The algorithm is executed on a processing device appropriately programmed using conventional computer programming techniques.

The typical diabetic profile shape 201 is often observed to start off at a higher fasting glucose concentration, rise to higher concentrations (typically above 180 mg/dL) often at a faster rate, maintain higher glucose concentrations for a longer period of time, and to take longer to return toward a normal physiological glucose concentration of 80 to 120 mg/dL. After the peak, the rate of decrease of the glucose concentration may be minimal versus a subject with IGT or with normal physiological glucose response.

The IGT profile shape 202 has a response that starts with normal fasting glucose levels, rises quickly to levels between 140-200 mg/dL, and then falls back to normal. However, the return to normal glucose concentration typically occurs with a slower negative rate of change compared to a normal physiological response.

A normal glucose response profile 203 has a shape that shows a slight increase in glucose levels to <140 mg/dL and generally returns within two hours to normal levels. The shape may be quite angular with very quick rates of glucose change indicating normal insulin function. The final segment of the profile is generally flat in the normal ranges.

Low glucose tolerance 204 (LGT) or hyperinsulinemia produces a shape or profile that starts with low to normal fasting glucose levels. The shape then shows a sharp increase in glucose response. The peak of the shape is usually dramatic, as glucose levels rarely linger in the elevated range. A shape with a peak at two hours might be indicative of a different phase two insulin response than that of a peak at three to four hours. The decrease continues through the normal range to blood glucose levels typically below 60 mg/dL. Hypoglycemia triggers the adrenergic response causing the shape of the response to rise again into normal ranges.

In a first embodiment of the invention, a simple comparison algorithm is provided that compares selected parameters from a subject's profile with predetermined thresholds for the various conditions. The thresholds may be determined from standard diagnostic criteria for the various conditions. For example, a diabetic has a fasting plasma glucose level greater than or equal to 140 mg/dL or a 2-hour post challenge glucose level greater than or equal to 200 mg/dL. A subject with impaired glucose tolerance has a fasting plasma glucose level less than 126 mg/dL and/or a 2-hour post challenge glucose level between 140 mg/dL and 200 mg/dL. A person with normal physiological tolerance has a fasting plasma glucose concentration of less than 140 mg/dL and/or a two-hour post challenge glucose concentration less than 140 mg/dL. An individual suffering from LGT typically has a fasting plasma glucose level less than 85 mg/dL and/or a 2-hour post challenge glucose level between 140 mg/dL and 200 mg/dL, and a 3-4 hour post challenge glucose level less than 70 mg/dL. Another example may be the area (glucose concentration multiplied by time) above a normal baseline of 80 mg/dL during the course of a glucose tolerance test. One species would be the area as determined by integrating area under a glucose perturbation and above an 80 mg/dL baseline during a specified time such as 60 minutes to 3 hours. Another example would be based upon the negative rate of change of the glucose concentration after the peak glucose concentration is obtained. A diabetic may have a decrease of only 20 mg/dL/hour while a normal physiological response may be 100 mg/dL/hour. The algorithm compares the values of the one or more of these parameters from the subject's profile with the predetermined thresholds, and on the basis of the comparison, classifies the profile (and thus, the subject) as normal, diabetic, having IGT, or having LGT. The above parameters are exemplary only. One skilled in the art will appreciate other parameters and combinations that are consistent with the spirit and scope of the invention.

Once a classification has been made (diabetic, IGT, normal), information about related diabetic diseases/symptoms may be presented to the subject. For example, if a subject is classified as having impaired glucose tolerance, then the subject would be made aware that they are at risk for heart disease, stroke, kidney disease, neuropathy, retinopathy, diabetic ketoacidosis, skin conditions, gum disease, impotence, and/or a shorter lifespan. The subject may be counseled to seek the advice of their healthcare practitioner.

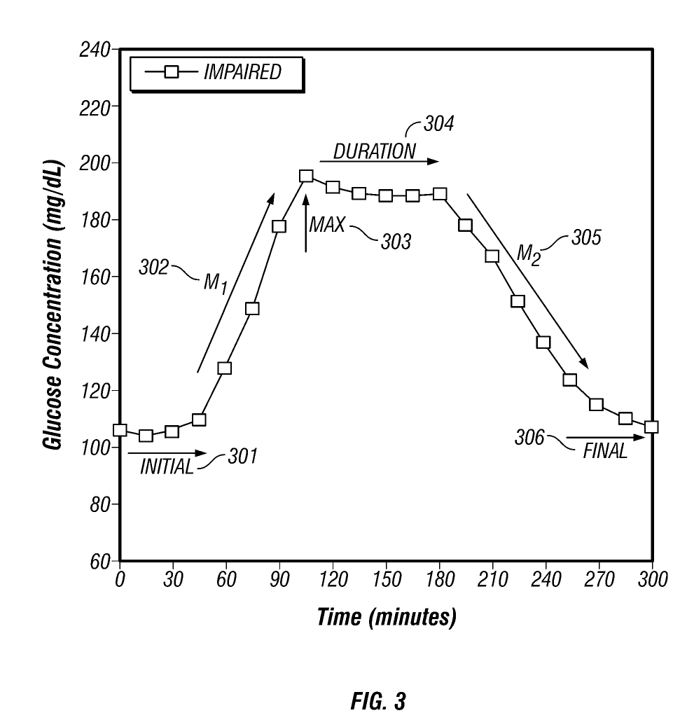

In an alternate embodiment, glucose concentration values as a function of time are input to a continuum mathematical algorithm that evaluates the series to determine if the range of values screens the subject as a diabetic, as having IGT, normal physiological function, or LGT. A number of parameters may be utilized individually or in combination to make this determination. Some of these parameters are identified in FIG. 2. Additional parameters are identified in FIG. 3.

The first parameter 301 is the initial glucose concentration (FIG. 3: Initial). An increased initial glucose concentration is diagnostic of diabetes. The ADA (American Diabetes Association) states that an initial fasting glucose concentration of greater than 126 mg/dL is an indication of diabetes. The ADA also states, in the absence of external insulin injections, a fasting glucose concentration less than 123 mg/dL is indicative of normal physiological function but could also be IGT. However, in this continuum algorithm more extreme numbers are assigned to a diabetic and normal state so that a range of weights from 0 to 1 can be assigned to intermediate levels. For example, a fasting glucose concentration >140 mg/dL is a very strong indication of diabetes and could be assigned a value of 1, as are all fasting glucose concentrations above 140 mg/dL. A fasting glucose concentration of 80 mg/dL is an indication of normal physiological function and could be assigned a value of 0, as are all glucose concentrations less than 80 mg/dL. A linear or nonlinear scale can then be applied between the two values. Thus, on a linear scale, a glucose concentration of 120 is assigned a weight of 0.66. This indicates a reasonable likelihood of IGT whereas a weight of 1 is indicative of diabetes and a weight of 0 is indicative of normal physiological function.

For LGT screening, a fasting glucose concentration less than 50 mg/dL is an indication of LGT and would be assigned a value of 0. A linear or non-linear scale can then be applied between the values of <50 mg/dL and 80 mg/dL. With a linear scale, a value of 65 mg/dL would be assigned a value of 0.55. Prior to an evaluation of LGT, additional parameters would be necessary. Alternately, a single scale can be employed to diagnose all conditions. In this case, a fasting glucose concentration of 50 mg/dL, indicative of LGT has a weight of 0, a normal blood glucose concentration of 80 mg/dL has a weight of 0.33 and the diabetic value of 140 mg/dL still has a weight of 1.

A second parameter 302 is the rate at which the glucose concentration rises (FIG. 3: m₁). In general, a higher slope is indicative of diabetes while smaller slopes indicate IGT and still smaller slopes are indicative of a normal physiological response. Initial slopes indicative of diabetes may range from 1 to 7 mg/dL/min; whereas, normal physiological function results in rates of change from 0 to 2 mg/dL/min. Intermediate rates are indicative of IGT. Due to the fact that the rates from each cluster overlap, only more extreme values could lead to an accurate classification, based on evaluation of the rate of change. As described above, high slopes (above 3 mg/dL/min) may be assigned a weight of 1 while low slopes (less than 0.5 mg/dL/min) may be assigned a value of zero. Again using a linear scale, a slope of 2.5 mg/dL/min would be assigned a weight of 0.8 and would be interpreted as a positive screening for diabetes.

A third parameter 303 is the maximum monitored glucose concentration (FIG. 3: max). Glucose levels peaking above 220 mg/dL are an indication of diabetes, and may be assigned a weight of 1. Only a slight rise above the high end of the normal glucose concentration of 120 mg/dL is indicative of normal physiological activity. Thus, glucose concentrations of 120 mg/dL or below may be assigned a weight of 0. Elevated but not grossly high glucose concentrations (160 to 220 mg/dL) are indicative of IGT and are then assigned intermediate weights. A positive correlation is known to exist between the diagnosis of normal, IGT, or diabetes with the peak glucose concentration monitored. This correlation is well known and accepted; therefore, this parameter may be given a larger weighting function.

A fourth parameter 304 is the duration that the glucose concentration remains elevated (FIG. 3: duration). The longer the duration above a given threshold, the more indicative the data are of diabetes. For example, 15 minutes above 200 mg/dL may indicate IGT while 1 hour above 200 mg/dL is indicative of diabetes.

A fifth parameter 305 is the rate of decrease of the glucose concentration after the peak glucose concentration (FIG. 3: m₂). Typically, the sharper the decrease, the more on the continuum the data is toward normal physiological function. As observed in FIG. 2, there exists an appreciable spread of rates of change after the peak glucose concentration for subjects ranging from diabetic to normal, making this parameter a particularly sensitive indicator for diabetes or for IGT. Thus, this parameter may then be given a larger weighting function.

A sixth parameter 306 is the minimum glucose concentration obtained after the maximum (FIG. 3: final). Glucose values that fall below 120 mg/dL without a dose of insulin are indicative of normal physiological response whereas glucose concentrations that stay above 150 mg/dL are indicative of diabetes. Glucose values that fall below 80 mg/dL could be indicative of LGT. As with the first parameter, values below 50 mg/dL would be assigned a value of 0 and at 150 mg/dL a value of 1.

One or more of these parameters may be utilized to determine if the subject is diabetic, has impaired glucose tolerance, has a normal physiological response, or low glucose tolerance according to equation 1, where SF is the screening factor, P₍₁₋₆₎ are parameters, and W₍₁₋₆₎ are weights:

$$S F = \frac{( {P}_{1} {W}_{1} + {P}_{2} {W}_{2} + {P}_{3} {W}_{3} + {P}_{4} {W}_{4} + {P}_{5} {W}_{5} + {P}_{6} {W}_{6} )}{( {W}_{1} + {W}_{2} + {W}_{3} + {W}_{4} + {W}_{5} + {W}_{6} )} \qquad ( 1 )$$

One or more of the parameters may be utilized to compute the screening factor and weights for each parameter may range from 0 to 1. Essentially, the screening factor is a weighted average of the individual scaled parameters. An average or a weighted final score can be computed from the individual score(s). Thresholds can then be determined to classify the subject into one of the three clusters. Any number of limits defining diabetic or non-diabetic may be established. Similarly linear or nonlinear axes may be established for any of the scores. These parameters may be established based on the most current diagnostic criteria provided by bodies such as, for example, the American Diabetes Association.

A seventh parameter 401 is the area under the curve representing the glucose excursion through time after a glucose challenge. The area under the curve may originate at the time of glucose intake or sometime in the first 30 minutes thereafter and continues until termination of the glucose challenge or until a period not less than one hour before termination of the profile. Typically, the glucose challenge lasts for 3 to 5 hours. As an example utilizing the glucose profiles presented in FIG. 3, the area under the curve as calculated by the summation of the observed difference between the observed glucose concentration and a baseline of 80 mg/dL, is 293, 1204, and 2020 for the normal, impaired, and diabetic profiles, respectively. If the limits of 300 and 2000 were utilized as the zero and one limits of the normalized continuum scale then the 1204 would read as 0.53 and be interpreted as IGT.

An eighth parameter is the area under the curve after the peak glucose concentration to an endpoint in time. It is recognized that the differences between the areas under the curve in this region would be more sensitive to the diagnosis of diabetes, IGT, or normal function due to the different negative rates of change of the glucose concentration 305 observed after the peak glucose concentration. An example follows from the glucose profiles presented in FIG. 3 that again calculates the summation of difference between the observed glucose concentrations and an 80 mg/dL baseline. The observed areas under the curve from 120 to 300 minutes are 41, 866, and 1573 for the normal, IGT, and diabetic profiles, respectively. The large spread between these areas allows for a sensitive metric in the classification of the glucose tolerance. This sensitivity is not lost upon normalization. Here, use of 100 and 1500 for the areas under the curve associated with the zero and one limits results in a value of 0.55 for the IGT profile presented.

Equation 1 utilizes only parameters introduced in FIG. 2. A similar equation for parameters seven and eight could be generated from parameters introduced in FIG. 3 as in equation 2, where SF₂ is the screening factor, P₍₇₋₈₎ are parameters, and W₍₇₋₈₎ are weights:

$$S {F}_{2} = \frac{( {P}_{7} {W}_{7} + {P}_{8} {W}_{8} )}{( {W}_{7} + {W}_{8} )} \qquad ( 2 )$$

It is recognized that a number of additional parameters may be readily constructed via mathematical manipulation or comparisons of the earlier parameters. For example, a representative ninth parameter may be the ratio of the area under the curve after a given point in time (8^{th} parameter) to the total area under the curve (7^{th} parameter) as in equation 3.

9^{th} parameter=8^{th} parameter/7^{th} parameter (3)

For example, a series of such parameters may be made via ratios or differences. While these parameters are not independent, some of them are more sensitive to the diagnostic issue at hand. It is further recognized that greater precision and sensitivity of combinations of parameters will not always result in a better diagnostic. For example, if the test is conclusive for IGT, a more sensitive test for IGT is not required.

Similarly, combinations of parameters from FIGS. 2 and 3 can be combined with or without mathematically generated parameters as in equation 4, where SF₃ is the screening factor, P₍₁₋ₙ₎ are parameters, and W₍₁₋ₙ₎ are weights:

$$S {F}_{3} = \frac{( {P}_{1} {W}_{1} + {P}_{2} {W}_{2} + {P}_{3} {W}_{3} + … + {P}_{n} {W}_{n} )}{( {W}_{1} + {W}_{2} + {W}_{3} + … + {W}_{n} )} \qquad ( 4 )$$

An example of a threshold screen limit is:

$$\begin{matrix}S {F}_{4} = \frac{( {P}_{1} {W}_{1} + {P}_{6} {W}_{6} )}{( {W}_{1} + {W}_{6} )} ; and & ( 5 ) \\ S {F}_{5} = \frac{( {P}_{2} {W}_{2} + {P}_{3} {W}_{3} + {P}_{4} {W}_{4} + {P}_{5} {W}_{5} )}{( {W}_{2} + {W}_{3} + {W}_{4} + {W}_{5} )} ; & ( 6 )\end{matrix}$$

where:
SF₄<0.25 and SF₅<0.1 indicates normal glucose tolerance;
0.25<SF₄<0.5 and 0.1<SF₅<0.16 indicates LGT;
0.5<SF₄<0.75 and 0.16<SF₅<0.325 indicates IGT; and
SF₄>0.75 and SF₅>0.325 indicates diabetes.

Any additional combination indicates the likelihood of a medical condition related to insulin and glucose tolerance exists, but is not readily defined in the individual's current physiological state. Such an outcome suggests a need for additional testing and evaluation by the individual's healthcare provider.

Other algorithms for providing the same information will occur to those skilled in the art and all are entirely within the scope of the invention. As the understanding of diabetes and diabetes screening increases, it is expected that the criteria set forth by the ADA and WHO will change, thus making it necessary to adjust the threshold values to meet current diagnostic criteria.

It is noted here that a complete glucose profile is not required for this approach to function. Missing data points can be overcome, as the data points are not independent from one another. Thus, some of the data from each parameter can be absent. In fact, if all of the data from some parameters is absent, the algorithm may still function by setting the weighting function for that parameter to zero. Inasmuch as glucose profiles tend to reproduce from day to day, partial data from each day can alternatively be utilized in the function. Although the precision of the screening factor decreases, use of historical data in place of a glucose or meal tolerance test helps to significantly minimize the pain and inconvenience entailed with invasive and minimally invasive glucose testing. In some cases, such as when a subject has kept good records of meal, glucose concentrations and/or insulin dosages, this data can be utilized as the input data, thus minimizing data collection time.

It should be recognized that all of the glucose concentrations may be collected prior to diagnosis. Therefore, parameters can be adjusted to fit the data. For example, in FIG. 3, the diabetic, IGT, and normal glucose responses peak at different elapsed times from a carbohydrate intake event. Because all of the data is available prior to diagnosis, algorithms such as area under the curve after the peak are not restricted to starting at particular times, but rather can start as the peak glucose response for any of the normal, impaired, or diabetic profiles.

Within a glucose profile, the individual data points are not independent, which makes it possible to determine outliers. Utilizing only a single individual glucose reading allows only gross outliers to be detected. For example, a glucose reading of 20 in a conscious subject is obviously an outlier. However, with multiple data points, small outliers may be determined. For example, if a series of glucose readings done at twenty-minute intervals is 80, 100, 120, 140, 160, 180, 142, 220, and 240 mg/dL then the data point 142 is readily determined to be an outlier. If a conventional two point test at fasting and at two hours were used, the 80 mg/dL would be the fasting value and the 142 mg/dL would be the two-hour value. Thus, the subject would have been screened as having a normal physiological glucose response, due to a value which, in actual fact was an outlier, when he or she was actually diabetic. In this way, the algorithm has built in safeguards against many of the hazards of poor screening.

The screening algorithm of equation 1 allows early detection of IGT. Complications associated with diabetes may thus be discovered earlier, allowing initiation of early treatment. Being made aware of the condition, which is largely due to environmental factors and to parameters such as body fat allows the individual to mitigate or prevent future diabetes-related complications. In settings where blood-borne pathogens are a risk, HIV clinics for example, this low-risk, bloodless approach to screening patients can be used to screen those who develop glucose abnormalities as a response to drug treatment therapies. The work place setting could use routine employee screenings for either glucose impairment or relative risk of complications.

The skilled practitioner will recognize that the inputs to the algorithms herein described are values of parameters that determine the shape of the glucose profile. It should be noted that a meaningful evaluation of profile shape is substantially a quantitative process, and that the shape of the profile is a function of the parameters and the corresponding values.

The above embodiments have dealt with obtaining actual values of blood glucose. As previously mentioned, screening based on relative blood glucose values is also possible. Advantageously, actual glucose concentrations are not required if relative glucose concentrations are available. Because it is the shape of the response that is utilized in the screening, differences in glucose concentration can be utilized to obtain a screening factor. For example, if a noninvasive or minimally invasive glucose testing procedure shows a relative increase in glucose concentration between the fasting level and the maximum concentration, then parameters 1 (fasting) and 3 (maximum) can be utilized to determine the screening factor without actual glucose concentrations.

Parameter 1 can be dropped (i.e. standardized to a predetermined value, for example 100 mg/dL), while Parameter 3 is adjusted to focus on the range of blood glucose values, rather than the maximum. Generally, individuals having normal glucose tolerance do not experience a change greater than 60 mg/dL, while someone suffering from IGT or LGT will see a change greater than 60 mg/dL, but unlikely to experience a change greater than 100 mg/dL. People suffering from diabetes often experience changes greater than 100 mg/dL. Thus the fuzzy logic would apply a weighting factor of 0 to a range of values <60 mg/dL, a weighting factor of 1 to a range of values greater than 100 mg/dL, and values ranging from 01 to 1 for glucose concentration between 60 and 100 mg/dL.

Parameter 6 then needs to be modified to account for LGT. This would be achieved by assigning a weighting factor of 0 to range values >−30 mg/dL from the standardized value and a weighting factor of 2 to a range values >30 mg/dL from the standardized value at the 3-4 hour mark of the tolerance test.

Subjects can be tested in obstetric settings for relative change in glucose concentration as an early screen for gestational diabetes. Actual numbers are not required, as the response or shape is easily identified as being that of an impaired response. As a result of detecting an impairment early, interventions such as dietary adjustments and self-monitoring of glucose are more likely to be effective. Additional time to schedule diagnostic procedures may be precious because the pregnancy may already be at a relatively advanced stage.

A further embodiment of the invention employs a pattern recognition system for the analysis of a glucose profile associated with a particular patient's OGTT (oral glucose tolerance test) to screen for disorders of glucose metabolism. This system has the advantage of high sensitivity and robustness with respect to uncertain and/or missing data. In addition to the measurement step described for the previous embodiments of the invention, the current embodiment preferably, but not necessarily, includes steps for processing, feature extraction, and classification.

### Processing

Preprocessing includes operations such as scaling, normalization, smoothing, derivatives, filtering and other transformations are designed to attenuate the noise or unwanted sources of variation and to perform corrections to the OGTT profile that enhance and make more accessible the signal of interest. The preprocessed measurement, [symbol] , is determined according to

y=h(t,x) (7)

where [symbol] is the preprocessing function, [symbol] is the glucose measurements and [symbol] is the vector of times associated with each glucose measurement. Useful processing steps include any of:
the detection of outliers through statistical and model based methods that exploit the properties of the profile;
autocorrelation;
non-causal filtering of the profile;
time series analysis and optimum filtering techniques (e.g., Kalman filtering);
phase and magnitude correction related to known error distributions between the measured profile and the reference glucose measurements;
mean-centering;
baseline correction;
normalization;
multivariate signal correction;
standard normal variate transformation;
calculating one or both of first and second derivatives of the profile; and
state transformations.

Multiple processing steps are generally performed and, in certain applications, the processed data are further treated by decomposition into abstract features such as principal components, wavelet basis components and Fourier coefficients.

In certain applications, the profile is enhanced through any of outlier analysis, filtering, and magnitude/phase correction prior to analysis by a physician or medical care provider. However, steps of feature extraction and classification preferably follow the processing of the OGTT profile. In this case, the use of first and second derivative steps is beneficial to the classification objectives.

### Feature Extraction

Feature extraction is any mathematical transformation that enhances a quality or aspect of the sample measurement for interpretation. The purpose of feature extraction is to concisely represent the information content of the data in the simplest and most accessible form prior to the application of the classification algorithm, thereby providing the greatest discrimination between various classes. The features are represented in a vector, [symbol] that is determined from the processed OGTT profile through

z=f(t,y) (8)

where f: [symbol] is a mapping from the measurement space to the feature space. Decomposing f(•) will yield specific transformations, fᵢ(•): [symbol] for determining a specific feature. The dimension, Mᵢ, indicates whether the i^{th} feature is a scalar or a vector and the aggregation of all features is the vector z. When a feature is represented as a vector or a pattern, it exhibits a certain structure indicative of an underlying physical phenomenon.

The individual features are divided into two categories:
abstract; and
simple.

Abstract features do not necessarily have a specific interpretation related to the physical system. Specifically, the scores of a principal component analysis are useful features although their physical interpretation is not always known. Simple features can be related directly to the processed profile. For example, the magnitude of the first and second derivative at key time points and the duration between various time points have been determined to be valuable features for classifying the nature and type of OGTT profile.

In addition, features can be derived from known information unrelated to the profile such as age, history of diabetes, weight, height, body mass index, gender, ethnicity, diet and exercise patterns, HbA1c levels, and insulin/c-peptide levels.

The compilation of the abstract and simple features constitutes the M-dimensional feature space. Due to redundancy of information across the set of features, optimum feature selection and/or data compression is applied to enhance the robustness of the classifier. Feature extraction often follows data preprocessing like mean centering, derivative transformations, smoothing, multiplicative signal corrections, and high and low pass digital filtering.

### Classification

The classification or categorization of subjects based on OGTT profiles and other electronic and demographic information can be approached using a wide variety of algorithms. From Bayesian classifiers that assume knowledge of statistical distribution information to nonparametric neural network classifiers that assume little prior information, a wide range of classifiers can be utilized to separate endocrine system function of individuals into groups. The decision rules can be defined by crisp or fuzzy functions and the classification algorithm used to define the decision rule can vary from a single decision point to a tree structure with progressive decision mechanisms on each layer.

While feature extraction determines the salient characteristics of measurements that are relevant for classification, the goal of the classification step is to determine the subject classification related to a particular disorder of glucose metabolism. In this step the patient is assigned a “normal” designation or one of a number of glucose metabolism disorders. Classification generally involves two steps: a mapping and a decision engine. The mapping measures the similarity of the features to predefined classes and the decision engine assigns class membership. In this section two general methods of classification are proposed. The first uses mutually exclusive classes and therefore assigns each measurement to one class. The second scheme utilizes a fuzzy classification system that allows class membership in more than one class simultaneously. Both methods require prior class definitions as described subsequently.

### Class Definition

The development of the classification system requires a data set of exemplar features from a representative sampling of the population. Class definition is the assignment of the measurements in the exploratory data set to classes. After class definition, the measurements and class assignments are used to determine the mapping from the features to class assignments.

Class definition is performed through either a supervised or an unsupervised approach. In the supervised case, classes are defined through known differences in the data. The use of a priori information in this manner is the first step in supervised pattern recognition which develops classification models when the class assignment is known.

Unsupervised methods rely solely on the exemplary set of features to explore and develop clusters or natural groupings of the data in feature space. Such an analysis optimizes the within cluster homogeneity and the between cluster separation. Clusters formed from features with physical meaning can be interpreted based on the known underlying phenomenon causing variation in the feature space.

A combination of the two approaches is used to utilize a priori knowledge, and exploration of the feature space for naturally occurring spectral classes. Under this approach, classes are first defined from the features in a supervised manner. Each set of features is divided into two or more regions and classes are defined by combinations of the feature divisions. A cluster analysis is performed on the data and the results of the two approaches are compared. Systematically, the clusters are used to determine groups of classes that can be combined. After conglomeration the number of final class definitions is significantly reduced according to natural divisions in the data.

Subsequent to class definition a classifier is designed through supervised pattern recognition. A model is created based on class definitions which transforms a measured set of features to an estimated classification. Since the ultimate goal of the classifier is to produce robust and accurate patient assessment an iterative approach must be followed in which class definitions are optimized to satisfy the specifications of the measurement system.

### Statistical Classification

The statistical classification methods are applied to mutually exclusive classes whose variation can be described statistically. Once class definitions have been assigned to a set of exemplary samples the classifier is designed by determining an optimal mapping or transformation from the feature space to a class estimate which minimizes the number of misclassifications. The form of the mapping varies by method as does the definition of “optimal”. Existing methods include linear discriminant analysis, SIMCA, k nearest-neighbor and various forms of artificial neural networks. The result is a function or algorithm that maps the feature to a class, c, according to

c=f(z) (9)

where c is an integer on the interval [1,P] and P is the number of classes.

### Fuzzy Classification

While statistically based class definitions provide a set of crisp classes, the patient-to-patient and day-to-variation in OGTT profiles change over a continuum of values and result in class overlap. It is therefore beneficial to provide a measure related to the extent to which a particular feature set is related to a given class. In addition, distinct class boundaries do not exist and many measurements are likely to fall between classes and have a statistically equal chance of membership in any of several classes. Therefore, “hard” class boundaries and mutually exclusive membership functions appear contrary to the nature of the target population.

A more appropriate method of class assignment is based on fuzzy set theory. Generally, membership in fuzzy sets is defined by a continuum of grades and a set of membership functions that map the feature space into the interval [0,1] for each class. The assigned membership grade represents the degree of class membership with “1” corresponding to the highest degree. Therefore, a sample can simultaneously be a member of more than one class.

The mapping from feature space to a vector of class memberships is given by

cₖ=fₖ(z) (10)

where k=1, 2, . . . P, fₖ(•) is the membership function of the kth class, cₖε[0,1] for all k and the vector [symbol] is the set of class memberships. The membership vector provides the degree of membership in each of the predefined classes.

The design of membership functions utilizes fuzzy class definitions similar to the methods previously described. Fuzzy cluster analysis can be applied and several methods, differing according to structure and optimization approach can be used to develop the fuzzy classifier. All methods attempt to minimize the estimation error of the class membership over a population of samples.

The invention finds application in healthcare facilities including, but not limited to: physician offices, hospitals, clinics, and long-term healthcare facilities. Alternatively, this technology could be utilized in public settings such as shopping malls and the workplace, or in private settings such as the subject's home.

Although the invention has been described herein with reference to certain preferred embodiments, one skilled in the art will readily appreciate that other applications may be substituted for those set forth herein without departing from the spirit and scope of the present invention. Accordingly, the invention should only be limited by the Claims included below.

1. A computer implemented method for screening a subject for disorders of glucose metabolism, comprising steps of:
measuring a glucose concentration profile using a glucose concentration analyzer, said glucose concentration profile comprising a plurality of blood glucose concentrations from at least after a glucose or meal challenge;
using a pattern recognition system to generate a screening factor, wherein said screening factor comprises a mathematical representation of at least a plurality of glucose concentrations within said glucose concentration profile, wherein said screening factor is uniquely associated with a state of glucose metabolism disorder, wherein said state of glucose metabolism disorder comprises a chronic condition, wherein said state of glucose metabolism disorder comprises a classification of any of:
a diabetic condition of diabetes mellitus, and
a pre-diabetic condition of diabetes mellitus; and
classifying the subject into one of said states of glucose metabolism disorder based on evaluation of said screening factor, wherein said screening factor comprises a representation of said glucose concentration profile; and
outputting said one of said states of glucose metabolism disorder to a display;
wherein said plurality of blood glucose concentrations comprises a time series.

2. The method of claim 1, wherein said blood glucose concentrations comprise actual values.

3. The method of claim 1, wherein said blood glucose concentrations comprise relative values.

4. The method of claim 1, wherein said screening factor is generated using a parameter, wherein said parameter is generated using an area under the curve of at least three glucose concentration of a glucose profile.

5. The method of claim 4, wherein said classifying step comprises:
comparing said screening factor with a corresponding predetermined value and/or a range of values indicative of either a normal condition or one of a plurality of abnormal conditions.

6. The method of claim 1, wherein said generating step further comprises the steps of:
determining a weight for each of a set of parameters,
wherein said step of determining a weight comprises assigning each of said set of parameters a value on a scale; and
wherein said scale corresponds to a predetermined threshold values for a normal condition and a diabetic condition, respectively.

7. The method of claim 1, wherein said mathematical representation is generated using at least four of:
an initial fasting glucose concentration;
a rate of increase of glucose concentration following said glucose challenge;
a peak monitored glucose concentration;
a duration glucose remains elevated;
a rate of decrease of glucose concentration following said peak concentration;
a minimum glucose concentration following said peak concentration;
an area under the curve for the glucose profile; and
an area under the curve during a subset in time of the glucose profile.

8. The method of claim 1, wherein said glucose concentration analyzer comprises a noninvasive glucose concentration analyzer.

9. The method of claim 1, wherein said glucose concentration analyzer comprises a minimally invasive blood glucose analyzer.

10. The method of claim 1, wherein said glucose concentration analyzer comprises an invasive blood glucose analyzer.

11. The method of claim 1, wherein said screening factor comprises a numerical value.

12. The method of claim 1, wherein said screening factor comprises representation of a shape of said glucose concentration profile.

13. The method of claim 1, wherein said screening factor comprises a result of an unsupervised classification, wherein said unsupervised classification uses an exemplary set of features to explore and develop clusters of data in feature space,
wherein said data comprises said glucose concentration profile.

14. A computer implemented method for screening a subject for disorders of glucose metabolism, comprising steps of:
measuring a glucose concentration profile using a glucose concentration analyzer, said glucose concentration profile comprising a plurality of blood glucose concentrations from at least after a glucose or meal challenge;
generating a screening factor, wherein said screening factor comprises a mathematical representation of at least a plurality of glucose concentrations within said glucose concentration profile, wherein said screening factor is uniquely associated with a state of glucose metabolism disorder, wherein said state of glucose metabolism disorder comprises a chronic condition, wherein said state of glucose metabolism disorder comprises a classification of any of:
a diabetic condition of diabetes mellitus, and
a pre-diabetic condition of diabetes mellitus; and
classifying the subject into one of said states of glucose metabolism disorder based on evaluation of said screening factor; and
outputting said one of said states of glucose metabolism disorder to a display,
wherein said screening factor comprises the result of a supervised classification, wherein said supervised classification defines a class of said screening factor through known differences in data, wherein data comprises said glucose concentration profile.
